(19) United States
(12) Patent Application Publication
(10) Pub. No.: US 2026/0345148 A1
(43) Pub. Date: Nov. 5, 2026

(54) QUICKSTITCH FIXATION SYSTEM

(71) Applicant: EndoFix Medical Technologies, Inc., Plainville, MA (US)

(72) Inventors: Rickey Hart, Wrentham, MA (US); William Blenkhorn, Medway, MA (US)

(21) Appl. No.: 19/197,675

(22) Filed: May 2, 2025

(51) Int. Cl. A61B 17/04; A61B 17/00; A61B 17/06

(52) U.S. Cl. CPC A61B 17/0401; A61B 17/0469; A61B 17/06166; A61B 2017/00862; A61B 2017/00867; A61B 2017/0409; A61B 2017/0414; A61B 2017/0446; A61B 2017/0475; A61B 2017/06052; A61B 2017/06185; A61B 2017/0619

(57) ABSTRACT

A method and a system to join edges of a tissue around a tear or defect along a vertical seam using single delivery curved needle configuration with two suture-based implants is described. The curved needle can be retracted into a straight shaft so that it can fit down a small diameter endoscope channel, and then extended to form a curved needle formed of nitinol. This eliminates the need to reload a fixation delivery assembly when multiple fixation points are required. Further, this configuration only requires two implants for up to four fixation points to enable cinching up a mattress stitch. The single delivery needle is configured to be preloaded with suture type implants with a slip knot placed around the first implant. The tailing suture from the slip knot passes through the delivery instrument. The entire anchoring system is configured to provide a running continuous suture stitch.

### FIELD

Illustrative embodiments of the invention generally relate to surgical tissue fixation and, more particularly, various embodiments of the invention relate to devices and methods of deploying the devices to fixate tissue.

### BACKGROUND

Surgical tissue to tissue fixation systems have primarily been manufactured from a variety of materials and typically contain multiple anchoring devices. Surgical Fixation devices have primarily been manufactured from stainless, titanium, non-absorbable plastic, absorbable plastic or some hybrid of plastic and ceramic. However, the use of flexible fixation devices is far less common.

There is a particular need in medical treatment settings of fixating soft tissues, such as closing gastroenterological defects, and for that matter devices with an adaptive, curved shape that could be used for any soft tissue to tissue approximation.

### SUMMARY OF VARIOUS EMBODIMENTS

In accordance with one embodiment of the invention, a method for joining edges of a tissue defect into a seam by implanting an anchor assembly connected via a single flexible strand for fixation of the tissue edges into a seam including puncturing a proximal surface of a tissue at a first fixation point with a delivery needle. The first fixation point is on a proximal side of the tissue defect.

The method further includes puncturing a distal surface of the tissue at a second fixation point with the delivery needle. The second fixation point is on a distal side of the tissue defect.

The method further includes extruding a first braided anchor segment of an anchor assembly from the delivery needle. The anchor assembly includes the single flexible strand, and at least two braided anchor segments slidably coupled to the single flexible strand. The single flexible strand passes through a bore hole that traverses from a proximal end to a distal end of the at least two braided anchor segments such that the proximal end of a first braided anchor segment is adjacent to a distal end of a second braided anchor segment.

The method further includes pulling the flexible strand a first time to deform the first braided anchor segment. The deformed first braided anchor segment is configured to prevent the first braided anchor segment from being pulled through the tissue.

The method further includes withdrawing the delivery needle from the second fixation point and the first fixation point.

The method further includes approximating the delivery needle at a third fixation point. The third fixation point is a first distance from the first fixation point.

The method further includes puncturing the proximal surface of the tissue at a third fixation point with the delivery needle. The third fixation point is on a proximal side of the tissue defect.

The method further includes puncturing the distal surface of the tissue at a fourth fixation point with the delivery needle. The fourth fixation point is on a distal side of the tissue defect, and the fourth fixation point a second distance from the second fixation point.

The method further includes extruding a second braided anchor segment from the delivery needle, and pulling the flexible strand a second time to deform the second braided anchor segment.

The method further includes withdrawing the delivery needle from the fourth fixation point and the third fixation point, and pulling the flexible strand a third time to cinch the flexible strand. Pulling the flexible strand draws the second fixation point toward the first fixation point and draws the fourth fixation point to the third fixation point to join edges of tissues into a seam.

The method further includes securing the flexible strand to keep the tissue edges joined and fixated by the anchor assembly, and cutting the flexible strand. The flexible strand is cut a distance away from the fourth fixation point.

Extruding the braided anchor segment may be accomplished by advancing a first drive tube to push the first anchor segment out of the delivery needle.

The plurality of braided anchor segments may include a slip knot placed around the flexible strand at the proximal end of the first braided anchor segment. Securing the flexible strand may include locking the flexible strand in place using a locking mechanism including at least one of a tag, a barb, a cinch, a toggled needle, or a distel knot. Securing the flexible strand may include a locking device or an overhand knot that can be run down to a fixation point.

Puncturing the distal surface of the tissue at a second fixation point with the delivery needle may follow continuously from puncturing the proximal surface of the tissue at the first fixation point with the delivery needle.

The first distance may be between about 5 mm and about 20 mm, and the second distance may be between about 10 mm and about 100 mm. The distance between the first fixation point, the second fixation point, the third fixation point, and the edge of the tissue defect is at least 5 mm for each of the fixation points. The first distance may be about equal to the second distance.

Extruding a first braided anchor segment may be performed by pushing the first braided anchor segment with a first drive tube. Extruding a second braided anchor segment may be performed by pushing the second braided anchor segment with a second drive tube.

In accordance with another embodiment of the invention, a surgical device for implanting at least two flexible braided anchor segments via a single flexible strand for fixation of the tissue edges into a seam including a handle assembly. The handle assembly has a cylindrical bore passing through the handle assembly on a center axis along a length of the handle assembly.

The surgical device further includes a curved delivery needle positioned in the cylindrical bore of the handle assembly. The curved delivery needle is hollow and has a common center line with the cylindrical bore of the handle assembly. The curved delivery needle includes an outer tube having a needle tip at a distal end of the curved delivery needle.

The surgical device further includes at least two braided suture segments slidably coupled to a flexible strand. The flexible strand is translationally threaded through each of the at least two braided suture segments, and the at least two braided suture segments are positioned in the curved delivery needle.

The surgical device further includes a first drive tube positioned in the delivery needle and having a common center line with the curved delivery needle. The first drive tube configured to extrude a first braided suture segment of the at least two braided suture segments.

The surgical device further includes a second drive tube positioned in the first drive tube and having a common center line with the first drive tube and the curved delivery needle. The second drive tube configured to extrude a second braided suture segment of the at least two braided suture segments. The at least two braided suture segments may include hollow braided suture segments.

The flexible strand may include a monofilament strand. The at least two braided anchor segments may be slidably coupled to the flexible strand. The flexible strand may pass through a bore hole that traverses from a proximal end to a distal end of each of the at least two braided anchor segments such that the proximal end of the first braided anchor segment is adjacent to a distal end of a second braided anchor segment. The at least two braided suture segments may include hollow braided suture segments aligned end to end having the monofilament strand threaded through the aligned at least two hollow braided suture segments.

A column strength of the at least two braided suture segments may be increased by application of at least one of heat or a cyanoacrylate coating to each of the segments of the plurality of hollow braided suture segments. The braided suture segments of the at least two braided suture segments may have a length of between about 5 mm and about 50 mm. Each of the braided suture segments of the at least two braided suture segments may have a length of between about 5 mm and about 12 mm.

A distal end of the flexible strand threaded through the first braided suture segment may be looped around the first braided suture segment and knotted around the flexible strand between the first braided suture segment and the first drive tube.

Each of the at least two braided suture segments may be treated to increase a rigidity of each of the at least two braided suture segments slidably coupled to the flexible strand. The treatment may include heating each of the at least two braided suture segments, or may include applying a cyanoacrylate coating to each of the at least two braided suture segments.

The curved delivery needle may include nitinol. The curved delivery needle may be flexible.

The handle assembly may further include an outer sheath. The outer sheath may be positioned in the cylindrical bore of the handle assembly. The curved delivery needle is slidably positioned in the outer sheath. When the curved delivery needle is withdrawn into the outer sheath, the curved delivery needle may conform to straight tube. When the curved delivery needle is pushed out of the outer sheath, the curved delivery needle may return to its curved shape.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1A shows a schematic plan view of two flexible braided anchor assemblies for tissue fixation in conjunction with two additional fixation points to form a “mattress stitch” according to one embodiment of the present disclosure.

FIG. 1B shows a schematic plan view of the two flexible braided anchor assemblies for tissue fixation in conjunction with the two additional fixation points to form a “mattress stitch” after the suture has been “cinched” to close the defect in the tissue according to one embodiment of the present disclosure.

FIG. 2A shows a schematic view of the curved nitinol delivery needle extended out of a delivery shaft and pierced through the tissues according to one embodiment of the present disclosure.

FIG. 2B shows a schematic view of the first anchor after being deployed on the proximal side of the tissue according to one embodiment of the present disclosure.

FIG. 2C shows a schematic view of the needle point being approximated a distance, D, away from the first fixation point according to one embodiment of the present disclosure.

FIG. 2D shows a schematic view of a seam of fixated tissue after a mattress stitch has been drawn together according to one embodiment of the present disclosure.

FIG. 2E shows a schematic view of the mattress stitch closure according to one embodiment of the present disclosure.

FIG. 3A shows a schematic view of the curved nitinol needle tip piercing through the proximal side of the tissue according to one embodiment of the present disclosure.

FIG. 3B shows a schematic view of the first implant being extruded from the needle tip according to one embodiment of the present disclosure.

FIG. 3C shows a schematic view of the first anchor located on the proximal side of the tissue according to one embodiment of the present disclosure.

FIG. 3D shows a schematic view of the deployed first anchor after the delivery needle has been removed according to one embodiment of the present disclosure.

FIG. 3E shows a schematic view of the fixation points positioned around the defect in the tissue with the mattress stitch in place prior to tightening the suture according to one embodiment of the present disclosure.

FIG. 4A shows a schematic cross section drawing of an embodiment of a distal end of a delivery assembly according to one embodiment of the present disclosure.

FIG. 4B shows another schematic cross section drawing of an embodiment of a distal end of the delivery assembly according to one embodiment of the present disclosure.

FIG. 4C shows a schematic drawing of the first and second implants outside of the delivery needle according to one embodiment of the present disclosure.

FIG. 4D shows another schematic cross section drawing of an embodiment of a distal end of a delivery assembly according to one embodiment of the present disclosure.

FIG. 4E shows another schematic cross section drawing of an embodiment of a distal end of a delivery assembly according to one embodiment of the present disclosure.

FIG. 4F shows another schematic drawing of an embodiment of a distal end of a delivery assembly according to one embodiment of the present disclosure.

FIG. 5 shows a schematic cross section of an embodiment of a delivery assembly according to one embodiment of the present disclosure.

FIG. 6 shows embodiments of the outer tube, the first drive tube, the second drive tube, and the two implants strung on a flexible filament according to one embodiment of the present disclosure.

FIG. 7 shows a top view of the delivery assembly according to one embodiment of the present disclosure.

FIG. 8 describes a method for joining edges of a tissue defect into a seam according to one embodiment of the present disclosure.

FIG. 9A shows a schematic illustration of an embodiment of braided suture segments on a suture according to one embodiment of the present disclosure.

FIG. 9B shows a schematic illustration of an assembly of braided anchor segments as they are deployed on the distal side of the defect according to one embodiment of the present disclosure.

FIG. 10A shows a schematic illustration of an embodiment of a long braided suture segment on a suture according to one embodiment of the present disclosure.

FIG. 10B shows a schematic illustration of a long braided anchor segment as it is deployed on the distal side of a tissue suture according to one embodiment of the present disclosure.

### DESCRIPTION OF ILLUSTRATIVE EMBODIMENTS

In illustrative embodiments, the present disclosure includes a method and a system to join edges of a tissue around a tear or defect along a vertical seam using single delivery curved needle configuration with two suture-based implants. The curved needle can be retracted into a straight shaft so that it can fit down a small diameter endoscope channel, and then extended to form a curved needle formed of nitinol. The present configuration eliminates the need to reload a fixation delivery assembly when multiple fixation points are required. Further, this configuration only requires two implants for up to four fixation points to enable cinching up a mattress stitch, in contrast to other fixation devices which include four fixation implants for multiple point fixation of tissue.

In illustrative embodiments, the present disclosure includes a single curved delivery needle configuration which can accompany two soft tissue fixation devices at once. The device addresses tissue to tissue fixation. In particular, the device provides for joining edges of a defect and/or tear in a tissue. The configuration eliminates the need to reload a fixation delivery assembly when more than one fixation device (e.g., implant) is required. Further, this configuration can include different variations of devices via: length; diameter; connectivity; function; and deployment. In embodiments, these presently disclosed fixation devices are typically manufactured from standard braided polyester suture that is cut into predetermined lengths.

This design would be preloaded with either a monofilament or a braided type suture (e.g., suture) with a slip knot placed on the proximal end of the distal fixation device. The tailing suture from the slip knot will pass through the first and second fixation devices. The suture will then pass completely through the delivery assembly where it can be coiled and placed onto a card for storage outside of the delivery assembly.

To deploy the fixation devices, a series of internal tubes are required. A curved needle tip would first pierce a tissue on a proximal surface of the tissue on a proximal side of the defect, passed under the defect, and then puncture up through the distal surface of the tissue, on the distal side of the defect, to the proximal side of the tissue. The first fixation device would be pushed out of the curved delivery needle via a first drive tube (e.g., a compensator). This first drive tube will then be locked in place inside the handle mechanism against the proximal end of the first fixation device, and the suture will be pulled to deform the fixation device. As the suture is pulled, it deforms the fixation device into an anchor. By running the suture from the proximal side of the defect to distal side of the defect, a first connection between the two sides of the defect is formed.

The curved delivery needle is then removed from the distal side of the defect, and then the proximal side of the defect, and the reapproximated to pierce the proximal surface of the on the proximal side of the defect. The curved delivery needle is reapproximated to puncture the tissue in another location, typically about 5 mm to about 20 mm away. After puncturing the proximal surface on the proximal side of the defect a second time, the curved delivery needle is passed under the defect again, and then punctured up through the distal surface of the tissue, on the distal side of the defect, to the proximal surface of the tissue. The second fixation device would be pushed out of the curved delivery needle via a second drive tube.

The suture is pulled again to deform the second fixation device. For the second fixation device, the second drive tube provides the resistance against the pulling of the suture. This second drive tube will then be locked in place inside the handle mechanism against the proximal end of the second fixation device, and the suture will be pulled to deform the fixation device. As the suture is pulled, it deforms the second fixation device into an anchor. Once the second fixation device is extruded from the curved delivery needle and the fixation device is deformed, the curved delivery needle is removed from the distal side of the defect and the proximal side of the defect.

After both fixation devices have been placed in the distal side of the defect, the suture outside of the of delivery handle is pulled taut cinches up the mattress stitch that has been placed around the defect. Now the delivery assembly can be removed, and a locking device (e.g., locking mechanism) such as a cinch or special knot can be pushed down to complete and lock the mattress stitch. Additional locking mechanisms that may be used include a tag, a barb, a cinch, a toggled needle, or distal knot. The remaining suture is cut.

Alternately an additional fixation device, as described above can be added to the delivery instrument such as to act as a locking device.

The fixation devices are advanced or driven by a handle assembly that employs an internal metering function of the devices for the predetermined length of each fixation device. The handle assembly controls the delivery tubes and locks when required.

In some embodiments, the fixation devices are a pair of hollow braided suture segments placed end to end over a flexible strand (e.g., monofilament, or a braided suture). The braided suture segments are stored in a tube and extruded out of the needle for delivery. The braided suture segments on the flexible strand (e.g., suture) has a certain column strength that is determined by a given composition and characteristics of the braided suture segments and the flexible strand. The column strength of a given pair of braided suture segments may be strengthened (e.g., stiffened) by treating the braided suture segments. Given braided suture segments may be strengthened by heating the braided suture segments. Given braided suture segments may also be strengthened by coating them with a cyanoacrylate coating.

The braided suture segments may be formed of soft materials such as yarns, fibers, filaments, strings, fibrils, strands, sutures, etc., or any combination of such materials. The soft materials may be synthetic or natural materials, or combinations of synthetic and natural materials, and may be bio-degradable or non-degradable within the scope of this disclosure. In one non-limiting embodiment, the anchor assembly is made exclusively of soft, suture-based materials.

Details of illustrative embodiments are discussed below.

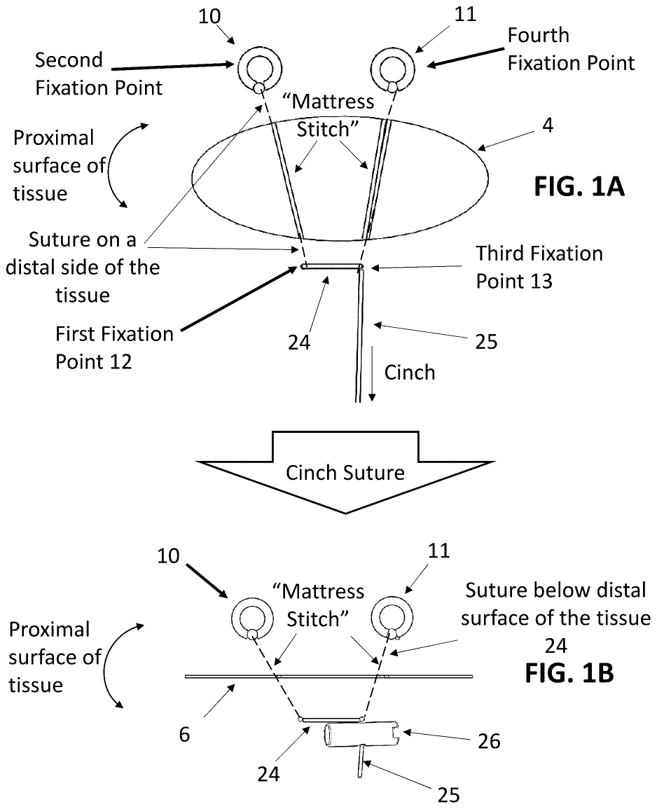

FIG. 1A shows a schematic plan view of two flexible braided anchor assemblies for tissue fixation in conjunction with two additional fixation points to form a “mattress stitch” in order to close a defect 4 (e.g., opening) in a tissue. A mattress stitch is formed between two fixation points on a first side (e.g., a proximal side) of a defect in a tissue 4, and two fixation points on second side (e.g., distal side) of the defect in the tissue 4. A puncture of the proximal surface of the tissue on the proximal side of the defect 4 with a suture 24 is a first fixation point 12. The first anchor 10 is a second fixation point on the proximal surface of the tissue on the distal side of the tissue defect 4.

A second puncture with the suture 24 is a third fixation point 13. A second anchor 11 is a fourth fixation point on the proximal surface of the tissue on the distal side of the defect 4. The first fixation point and the third fixation point 13 are joined by the suture 24, which maintains the fixation on the proximal surface of the tissue on the proximal side of the defect 4.

A single suture 24 filament is used to form all four fixation points, including positioning the first anchor 10 and second anchor 11. The mattress stitch is achieved using a single suture 24 to place the fixation points and span the defect 4 in the tissue. A suture tail 25 extends past the third fixation point 13. The suture tail (e.g., tailing suture) extends up through the delivery assembly in endoscope and exits the delivery assembly. The tailing suture 25 is used to cinch the mattress stitch closed.

FIG. 1B shows a schematic plan view of the two flexible braided anchor assemblies for tissue fixation in conjunction with the two additional fixation points to form a “mattress stitch” after the suture has been “cinched” to close the defect in the tissue. The suture tail 25 is pulled to cinch first anchor 10 to the second fixation point 12, and pull the second anchor 11 to the fourth fixation point 13. The defect 4 in the tissue is closed by shortening the suture 24 on the distal side of the tissue forming a horizontal seam of fixated tissue 6. After cinching the suture tail 25, a locking device 26 can be used to secure the suture tail 25, thus causing a tension in the mattress stitch and fixating the tissue in a seam.

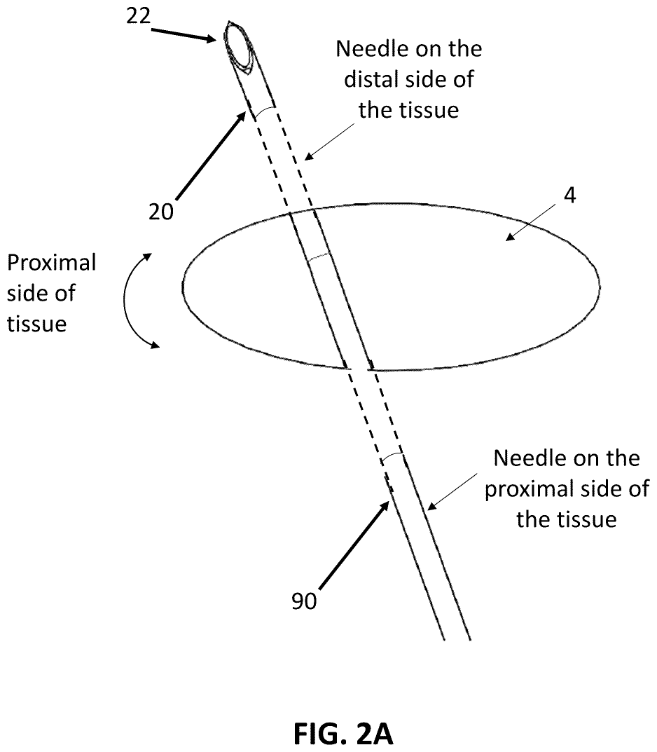

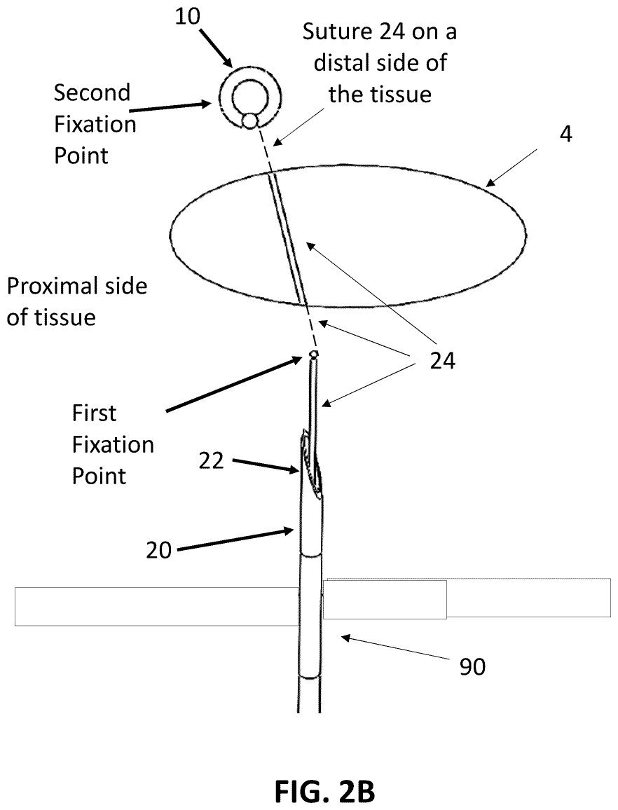

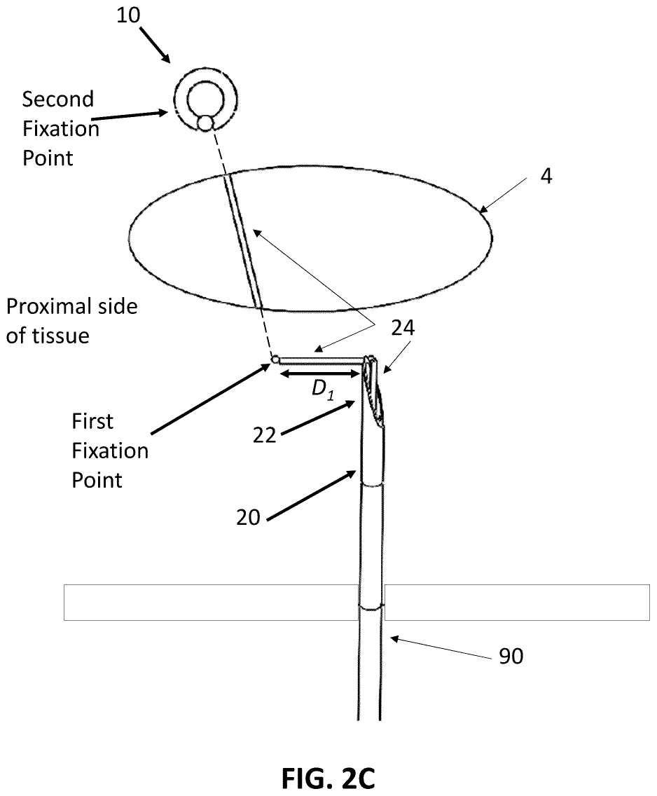

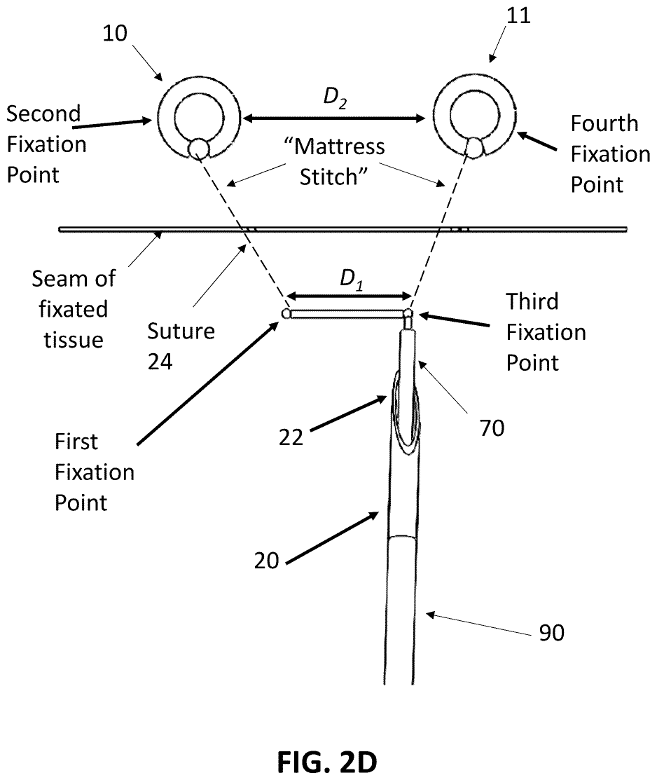

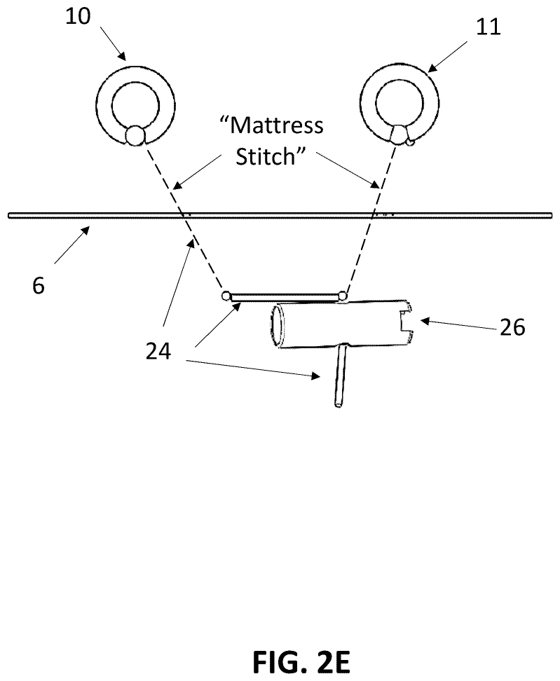

FIGS. 2A through 2E illustrate various steps in a method to fixate edges of a tissue into a seam. That is, the method joins the edges of a tissue to repair the tissue, and/or to join the edges of a defect in the tissue. The edges of the tissue are drawn together by a “mattress stitch.”

To deploy the fixation device, a preformed curved nitinol needle is disclosed for access and ease of placement. Once the instrument tip has been placed down through the endoscope, the needle tip is pushed distally which exposes the curved needle surface.

FIG. 2A shows a schematic view of the curved delivery needle 20 extended out of an outer tube 90 and pierced through the tissues. The needle tip 22 is pushed through the proximal surface of the tissue on the proximal side of the defect 4, passed under a defect 4 in the tissue, and then pushed up through a distal surface of the tissue of the distal side of the defect 4. The curved needle 20 allows for piercing down through the proximal surface of the tissue and then piercing up through the distal surface of the tissue.

In some embodiments, the needle 20 and the outer tube 90 may be fabricated as a single piece, whereas in other embodiments, the needle 20 and the outer tube 90 may be assembled from separate pieces. The needle 20 and the outer tube 90 may be fabricated from one or more rigid materials, such as a steel, aluminum, titanium, and so on. The rigid material may also include metallic alloys such as nitinol. In embodiments, when the needle 20 and the outer tube 90 are assembled from separate pieces, the needle 20 and the outer tube 90 may be fabricated from different rigid materials.

FIG. 2B shows a schematic view of the first anchor 10 after being deployed on the proximal side of the tissue. A first implant was deployed on the proximal side of the tissue, and then the suture was pulled to transform the first implant to the first anchor 10. The first anchor 10 provides a second fixation point. The suture 24 is shown by a dashed line as it passes under the tissue and as a solid line on the proximal side of the tissue and as it is visible in the defect in the tissue.

As the suture 24 passes through the proximal surface of the tissue on the proximal side of the defect, it forms a first fixation point. The suture 24 is visible in emerging from the needle point 22 as it is threaded through the needle 20.

In embodiments, the flexible strand (e.g., suture) 24 may be a monofilament. In other embodiments, the flexible strand 24 may be a suture. The suture 24 may be a braided polyester suture. In embodiments, the suture 24 may be a FiberWire®, TigerWire®, or FiberChain® suture, although any type of suture 24 may be utilized, including cored or coreless sutures. In another embodiment, the flexible strand 24 may be suture tape, such as FiberTape®. The flexible strand 24 may include any soft, flexible strand of material.

In some embodiments, the braided suture segments (e.g., implants) may be strengthened by heating the braided suture segments. The braided suture segments may also be strengthened by coating them with a cyanoacrylate coating.

FIG. 2C shows a schematic view of the needle point 22 being approximated (e.g., positioned) a distance, D₁, away from the first fixation point. The needle point 22 is positioned a distance, D₁, away from the first fixation point in preparation for the needle piercing the third fixation point on the proximal side of the tissue. The distance, D₁, between the first fixation point and the position on the proximal of the third fixation point on the proximal side of the tissue where the needle point 22 will puncture the tissue may be between about 5 mm and about 20 mm.

FIG. 2D shows a schematic view of a seam of fixated tissue after a mattress stitch has been drawn together. The mattress stitch is formed by having a suture 24 connecting the first anchor 10 to the first fixation point, and having the suture 24 connecting the second anchor to the third fixation point. The first anchor 10 and second anchor 11 provide anchor points against which the suture 24 stitches may be pulled against, and are separated by a distance, D₂. The distance, D₂, between the first anchor 10 and second anchor 11 may be between about 10 mm and about 100 mm. The anchors prevent the mattress stitch (e.g., suture) from being pulled through the tissue so that the two edges of the defect in the tissue 4 can be drawn together to fixate the tissue into a vertical seam.

A second drive tube 70 is shown extending from the needle tip 22. The second drive tube 70 is configured to push the second implant 11 out of the needle 20 once the needle has been positioned on the proximal side of the tissue at designated position.

FIG. 2E shows a schematic view of the mattress stitch closure after the locking device 26 has been secured to fixate the tissue by locking the suture 24 to prevent the suture from being pulled back below the surface to the distal side the tissue. Placing the mattress stitch has been completed with the first anchor 10 and second anchor 11.

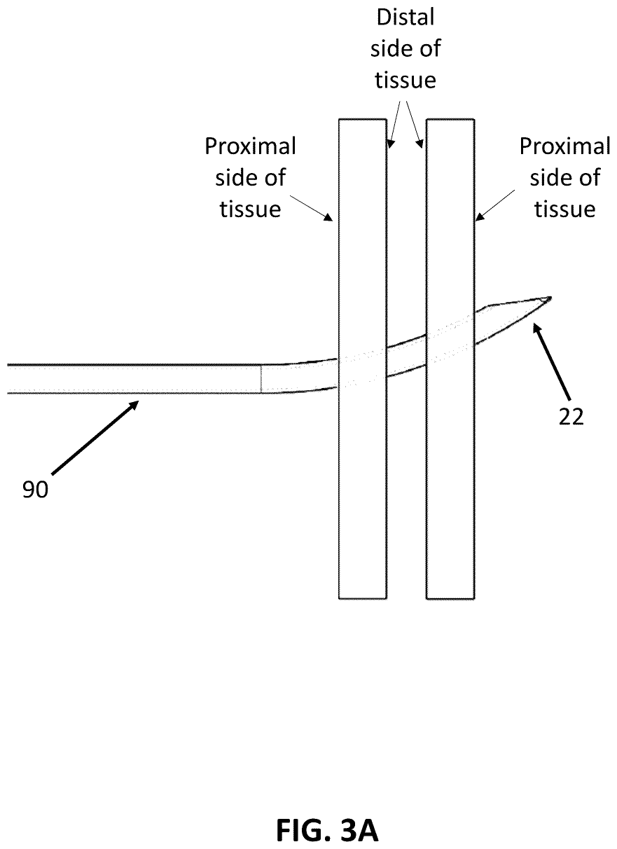

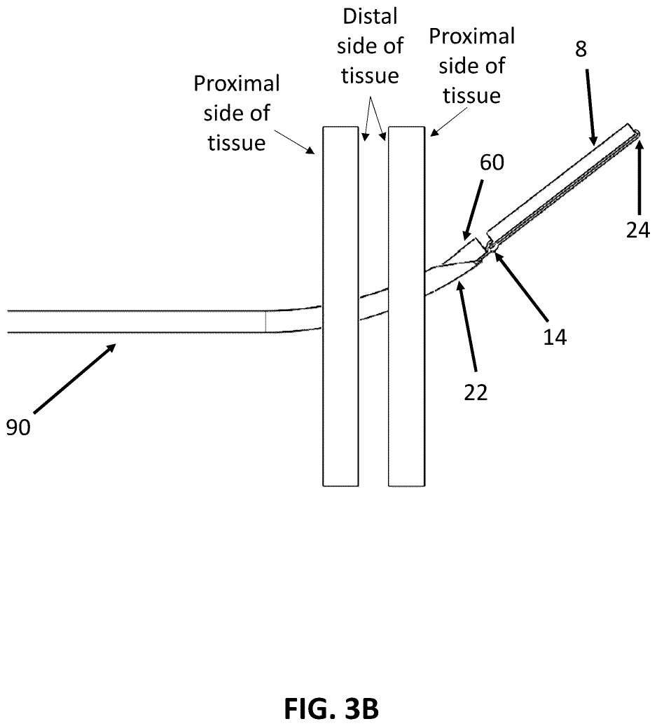

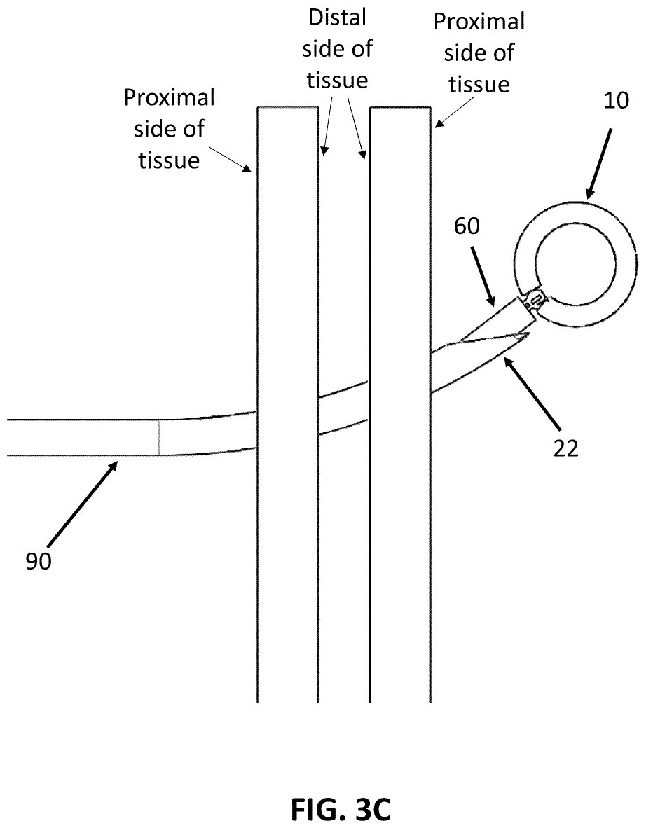

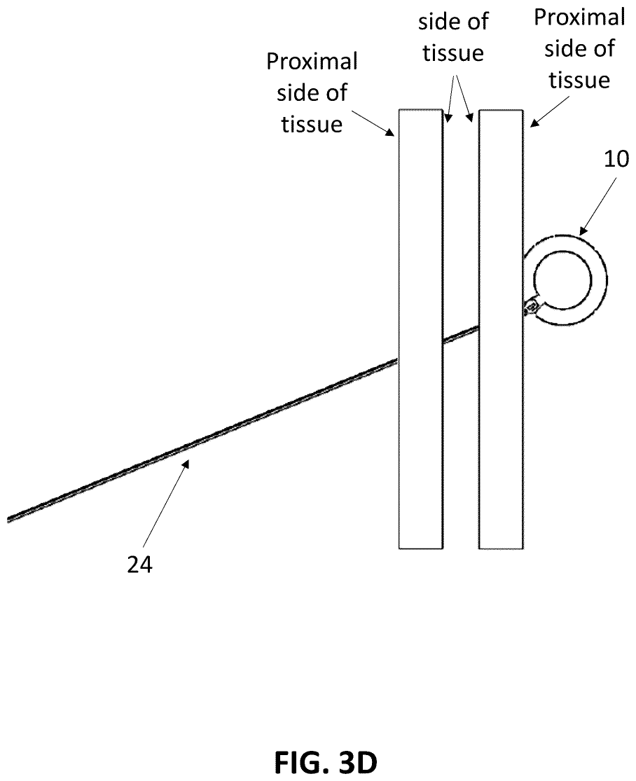

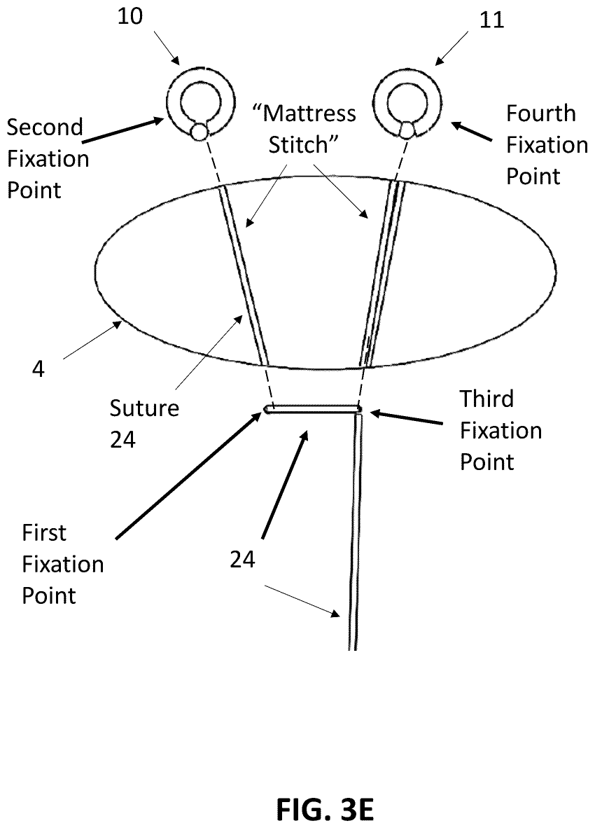

FIG. 3A shows a schematic view of the curved nitinol needle tip 22 piercing through the proximal side of the tissue to be repaired to the distal side. Then, the needle tip 22 pierces the distal side of the tissue to be repaired through to the proximal side. The curved design of the needle tip 22 allows for the same needle to be used to pierce through the proximal side of a tissue and curve around under the distal side of the tissue and then back though the distal side to exit the proximal side. This feature is also illustrated in the modified plan view of FIG. 2A.

FIG. 3B shows a schematic view of the first implant 8 being extruded from the needle tip 22. The suture 24 passes through the hollow first implant 8 from a proximal end of the first implant 8 to the distal end, where it doubles back to form a slip knot 14 with the suture 24 between the first implant 8 and the first drive tube 60. The first drive tube 60 pushes the first implant 8 from the needle tip 22 and is shown emerging from the needle tip 22.

The needle tip 22 first pierces the tissue before the defect, proximally and then continues to pierce through the defect and exit distally. The first implant 8 and the slip knot 14 are pushed out of the delivery needle 20 via the first drive tube (e.g., compensator) 60 after the needle tip 22 is positioned at the proximal surface of the distal side of the defect 4.

FIG. 3C shows a schematic view of the first anchor 10 located on the proximal side of the tissue on the distal side of the tissue defect. The first drive tube 60 then temporarily locks in place via a handle mechanism and the proximal end of the suture exiting the handle is pulled to deform the implant, thus forming the first anchor 10.

FIG. 3D shows a schematic view of the deployed first anchor 10 after the delivery needle 20 has been removed. The suture 24 is pulled taut and the first anchor 10 is firmly held against the proximal surface of the tissue to be repaired on the distal side of the repair.

Following the removal of the delivery needle 20 from the second fixation point and the first fixation point, the needle tip 20 is reapproximated 5 mm-20 mm away from the first fixation point to pierce the tissue defect in another location.

Then, similar to the first tissue piercing, the needle tip 22 is pushed through the tissue on the proximal side of the defect 4, through the defect and exits on the proximal surface of the tissue on the distal side of the defect 4. The second implant 11 is then pushed out of the needle tip 20 and out of the first drive tube via the second drive tube 70. The second drive tube 70 will then lock in place via the handle mechanism and the proximal suture exiting the handle is pulled to slightly deform the implant. A distance between the second fixation point and the fourth fixation point is a second distance and the second distance is between about 5 mm and about 20 mm. Furthermore, the second fixation point and the fourth fixation point are located at least 5 mm from the edge of the defect. Carefully, the instrument is removed from the defect 4 and approximated in front of the second needle entry location.

FIG. 3E shows a schematic view of the fixation points positioned around the defect 4 in the tissue with the mattress stitch in place prior to tightening the suture 24. The suture 24 is pulled to tighten the entire construct utilizing the second drive tube 70 as a backstop to pull against. This prevents pull through of the implants (e.g., anchors). After cinching the mattress stitch, the needle 20 is retracked into the outer sheath 34 and locked in place in the retracted position to protect the suture 24 from the needle bevel. Then, the delivery assembly can be safely removed from the endoscope. A suture locking device 26 can be threaded onto the suture 24 outside of the delivery device and run down the endoscope channel for deployment at the third fixation point. By deploying the suture locking device 26, the entire construct can be locked in place, and then the suture 24 can be cut.

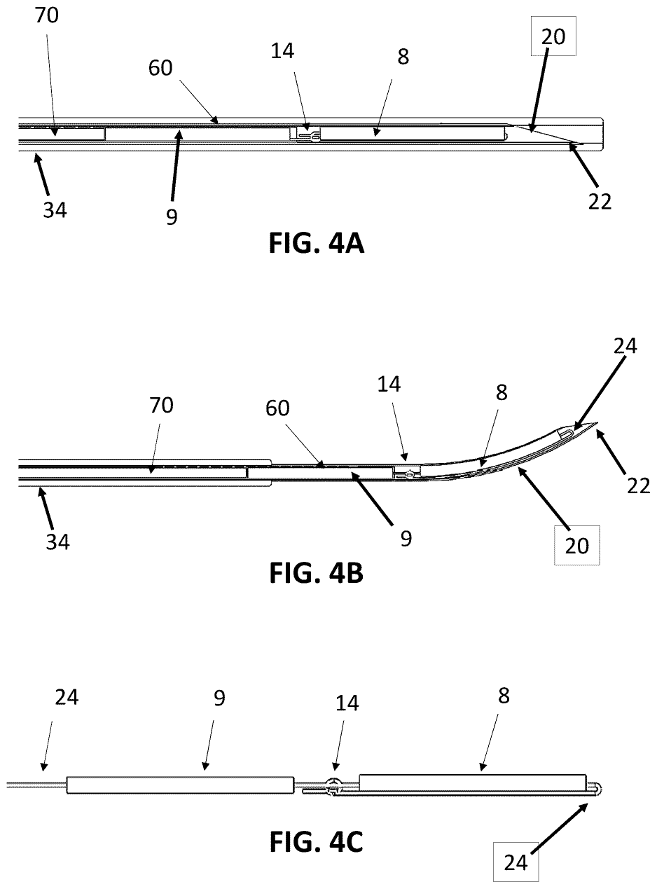

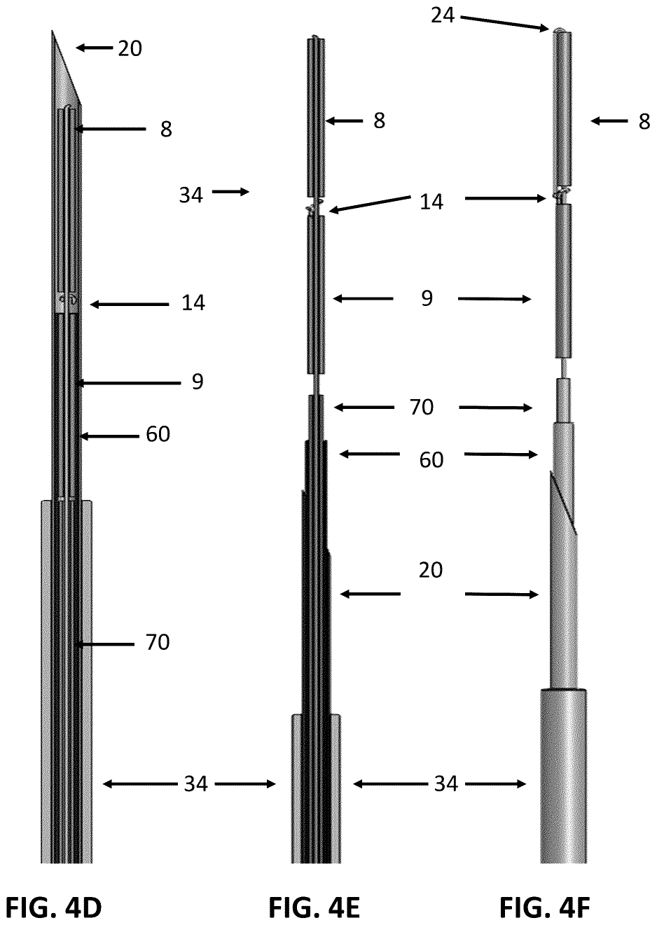

FIG. 4A shows a schematic cross section drawing of an embodiment of a distal end of the delivery assembly with the needle tip 22 withdrawn into the outer sheath 34. The needle tip 22 of the curved delivery needle 20 may be comprised of nitinol and is capable of being pulled into the outer sheath 34 and conforming to the straight cylindrical surface of the outer sheath. This flexibility of the delivery needle 20 allows for the sharp needle tip 22 to be retracted into the outer sheath 34 so that the delivery needle 20 can be inserted into the endoscope and pushed down endoscope passage to the region where the tissue repair will be performed. The delivery needle 20 with the sharp needle tip 22 is retracted into the outer sheath 34. In the retracted position, the outer sheath can be inserted into the endoscope and pushed down endoscope passage to the region where the tissue repair will be performed.

It is a surprising result that a curved delivery needle with a surgically sharp needle tip 22 can be maneuvered down a narrow endoscope passage to the surgical site without concern over damaging the endoscope or unintentionally cutting the patient. The specific design of the needle and the use of nitinol, or another flexible metal material enables the curved delivery needle 20 and needle tip 22 to be pulled into an outer sheath 34.

FIG. 4A also shows the second drive tube 70, the first drive tube 60, the slip knot 14, and the first implant 8 inside the outer tube of the curved delivery needle 20.

FIG. 4B shows a schematic cross section drawing of an embodiment of a distal end of the delivery assembly with the needle tip 22 pushed outside of the outer sheath 34. The curvature in the delivery needle 20 is apparent once the delivery needle 20 is outside of the outer sheath 34. The first drive tube 60 is in position to push the first implant 8 and the slip knot 14 out of the needle tip 22. The suture 24 is wrapped around the distal end of the first implant and forms the slip knot 14 between the proximal end of the first implant 8 and the distal end of the first drive tube.

FIG. 4C shows a schematic drawing of the first and second implants outside of the delivery needle. The suture 24 (e.g., flexible strand) passes through a bore hole that traverses from the proximal end to the distal end of each of the implants, such that the proximal end of the first implant 8 is adjacent to a distal end of the second implant 9. The sliding slip knot 14 is tied between the proximal end of the first implant 8 and the distal end of the second implant 9. Pulling the suture 24 the first time after the first implant 8 is positioned on the tissue surface tightens the slip knot 14 around a proximal end of the first implant 8. Tightening the slip knot is configured to distort the first implant 8 into the first anchor 10 in order to prevent the first implant 10 from pulling through the tissue.

FIG. 4D shows a schematic cross section drawing of the delivery assembly 30, FIG. 4E shows another schematic cross section the delivery assembly 30, and FIG. 4F shows a schematic drawing of the delivery 30. The delivery assembly 30 includes the needle 20 positioned concentrically in the outer sheath 34. The first drive tube (e.g., compensator) 60 is slidably positioned concentrically in the needle 20, and the second drive tube 70 is slidably positioned concentrically in the first drive tube 60. The suture 24 is threaded completely through the delivery assembly 30 from the proximal end through the first implant 8 at the distal end of the delivery assembly 30. The suture 24 is strung around the distal end of the first implant 8 and ties off into the slidable knot 14 between the proximal end of the first implant and the distal end of the second implant.

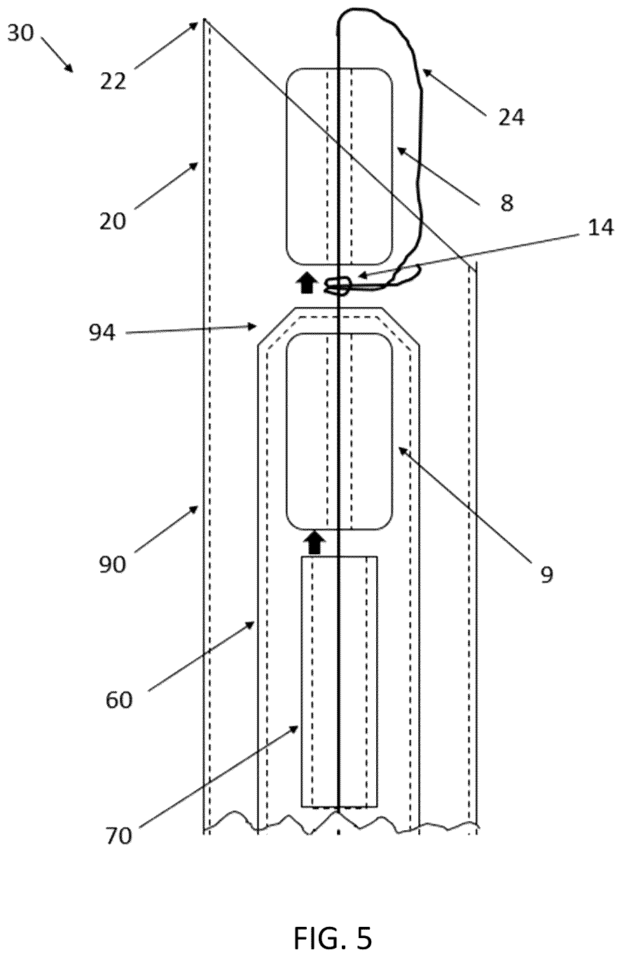

FIG. 5 shows a schematic cross section of an embodiment of a delivery assembly 30. The outer tube 90 has a delivery needle 20 with a needle tip 22. the first drive tube 60 is positioned concentrically inside of the outer tube 90. First drive tube 60 has an outer diameter that is smaller than the inner diameter of outer tube 90, and first drive tube 60 is used to push first braided suture segment 8 out of the distal end outer tube 90 through the delivery needle 20. In embodiments, a distal end of the first drive tube 60 may include a detent 94 that may provide friction so that there is a slight resistive force to the extrusion of the second braided suture segment 9. Once the first drive tube 60 has extruded the first suture segment 8, the first drive tube 60 may become temporarily fixed in the outer tube 90.

The second braided suture segment (e.g., implant) 9 is extruded from the first drive tube 60 as it is pushed by second drive tube 70. The second implant 9 is positioned in the first drive tube 60.

The suture (e.g., flexible strand) 24 may be threaded through implants and looped around the first implant 8 and tied into a slip knot 14 around the suture 24 between the proximal end of the first implant 8 and the distal end of the first drive tube 60.

A second drive tube 70 is positioned concentrically in the first drive tube 60 with its distal end proximal to the second implant 9. The second drive tube 70 has an outer diameter that is smaller than the inner diameter of first drive tube 60, and second drive tube 70 is used to push the second implant 9 out of the distal end first drive tube 60. In general, the second drive tube 70 is positioned proximal to the proximal end of the second implant 9 in the first drive tube 60.

In some embodiments, the delivery assembly may not include a first drive tube 60 (not shown). In embodiments, the braided suture segments 8 and 9 may be preloaded into an outer tube 90, and extruded by pushing the braided suture segments 8 and 9 out of outer tube 90 with the second drive tube 70. The braided suture segments 8 and 9 may be press fit into the detent 94 in the first drive tube 60 to provide friction to the sliding of the braided suture segments 8 and 9 against the pressure applied by the second drive tube 70.

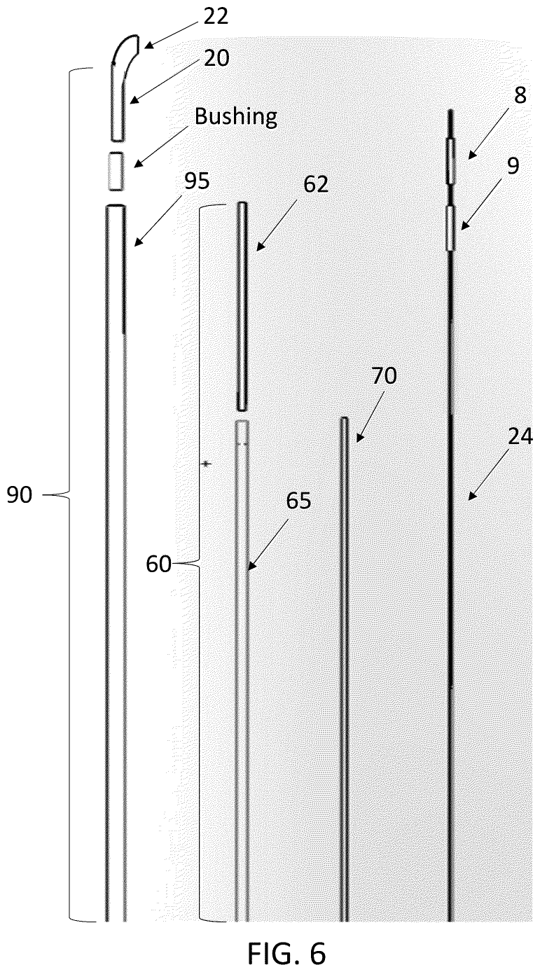

FIG. 6 shows embodiments of the outer tube 90, the first drive tube 60, the second drive tube 70, and the two implants strung on a flexible filament (e.g., suture) 24.

In some embodiments, the outer tube 90 is a single tube that includes a base outer tube 95, and a needle 20 having a needle point 22. In some embodiments, the single tube of the outer tube 90, may or may not have a bushing included. In some embodiments, the outer tube 90 may comprise a two-piece tube having a base outer tube 95 coupled with the needle 20. In some embodiments, the outer tube 90 may comprise a three-piece tube having the base outer tube 95 coupled with a bushing and the needle 20. In embodiments when the outer tube 90 comprises more than one part, the various parts may be fabricated from different rigid materials. In other embodiments, when the outer tube 90 comprises more than one part, the various parts may be fabricated from similar rigid materials.

FIG. 6 also shows embodiments of the first drive tube 60. In some embodiments, the first drive tube 60 is a single tube. In some embodiments, the first drive tube 60 may comprise a two-piece tube having a base first drive tube 65, and a first drive tube tip 62. In embodiments when the first drive tube 60 comprises more than one part, the various parts may be fabricated from different rigid materials. In other embodiments when the first drive tube 60 comprises more than one part, the various parts may be fabricated from similar rigid materials.

FIG. 6 further shows embodiments of the second drive tube 70. The second drive tube 70 is made from rigid materials. The rigid materials may include one or more rigid materials, such as a steel, aluminum, titanium, and so on. The rigid material may also include metallic alloys such as nitinol.

FIG. 6 still further shows embodiments of the assembly of two braided suture segments 8 and 9 with a flexible filament (e.g., suture) 24 threaded through bore holes that go through a length-wise dimension of the braided suture segments.

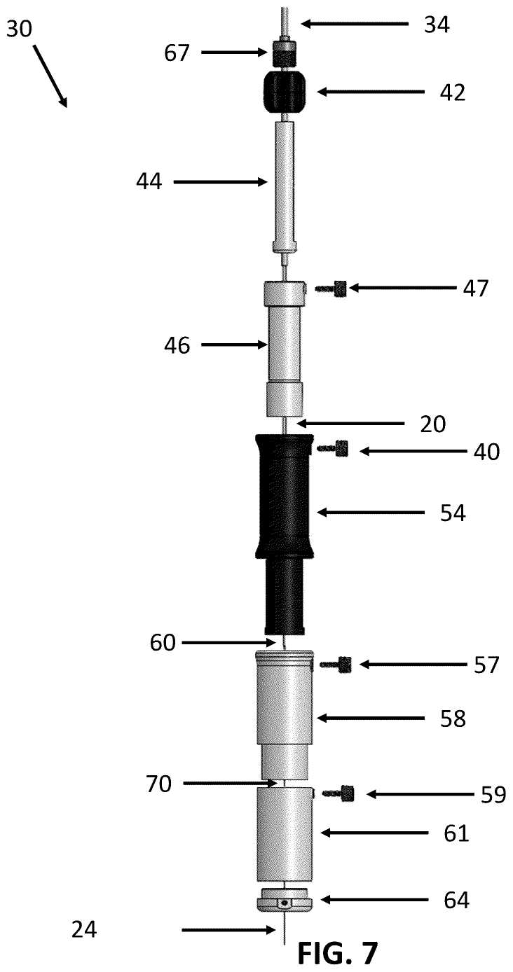

FIG. 7 shows a top view of the delivery assembly 30 according to one embodiment of the present disclosure. The suture 24 is threaded through the center bore through the delivery assembly 30 from the endcap 64 through the second implant 9 and first implant 8 that are positioned in the needle 20 at the distal end of the delivery assembly 30. The needle 20 is concentrically positioned in the outer sheath 34. The outer sheath 34 is mounted in a swivel that allows the outer sheath 34 to rotate to accommodate the orientation of the needle 20 inside the body. The outer sheath 34 is mounted in the scope attachment shaft 44, which itself is mounted in the shaft 46. The scope attachment shaft 44 is slidably mounted in the shaft 46 and the scope attachment shaft 44 has depth line mark indicators that can be adjusted to control the length of the outer sheath 34 that extends beyond the distal tip of the endoscope. The length of the outer sheath 34 can be locked at a given depth with the shaft thumb screw 47 at the distal end of the shaft 46.

The delivery needle 20 is positioned concentrically in the outer sheath 34 which is in the scope adjustment shaft 44. The shaft 46 is concentrically mounted in a needle drive handle 54 (e.g., front handle). The shaft 46 is used to push and pull the delivery needle 20 as it slides in and out of needle drive handle 54. The shaft 46 can be locked into position by tightening the needle drive handle locking thumb screw 40. The needle drive handle locking thumb screw 40 allows for the needle 20 to be locked either inside the outer sheath 34, or deployed out of the outer sheath 34 for surgical use.

A first drive tube pusher 58 (e.g., middle handle) with a first drive tube pusher locking thumb screw 57 is concentrically mounted on the needle drive handle 54. The first drive tube pusher 58 can be used to independently control the first drive tube 60 to push the first implant 8 out of the delivery needle 20.

A second drive tube pusher 61 (e.g., middle handle) with a second drive tube pusher locking thumb screw 59 is concentrically mounted on the first drive tube pusher 58. The second drive tube pusher 61 can be used to independently control the second drive tube 70 to push the second implant 9 out of the delivery needle 20. An endcap 64 is mounted on the proximal end of the second implant pusher 61.

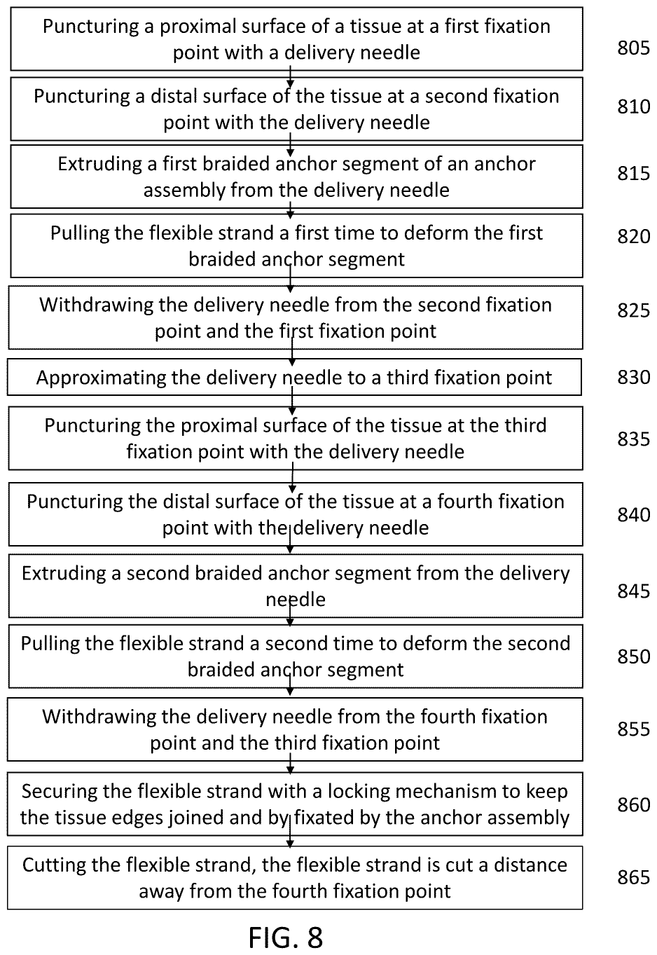

FIG. 8 describes a method for joining edges of a tissue defect into a seam.

At 805, a proximal surface of a tissue is punctured at a first fixation point on a proximal side of a defect 4 with a delivery needle 20. The delivery needle 20 punctures down through the proximal surface of the tissue.

At 810, a distal surface of the tissue is punctured at a second fixation point on the distal side of the defect 4 with the delivery needle 20. The curved delivery needle 20 is turned to press through the distal surface of the tissue on the distal side of defect 4 to the proximal surface of the tissue.

At 815, a first braided anchor segment 8 of an anchor assembly is extruded from the delivery needle 20 at the proximal surface of the distal side of the defect 4. The first braided anchor segment (e.g., implant) is pushed out of the delivery needle 20 by the first drive tube 60.

At 820, the flexible strand 24 (e.g., suture) is pulled a first time to deform the first braided anchor segment 8. The suture is pulled and the distal end of the first drive tube 60 provides resistance against the proximal end of the first implant 8 to deform the implant into an anchor, and prevents the first anchor 10 from pulling through the tissue. The first drive tube 60 is then retracted back into the needle.

At 825, the delivery needle is withdrawn from the second fixation point and the first fixation point. Following the pulling of the suture 24 and the deforming of the implant, the delivery needle 20 is retracted back from the first and second fixation points. At this point the sharp edge of the needle tip is exposed, but there is not any danger of the sharp edge cutting the suture.

At 830, the delivery needle 20 is approximated to a third fixation point. The delivery needle 20 is positioned on the proximal surface at a third fixation point. A first distance between the third fixation point and the first fixation point is between about 5 mm and about 20 mm.

At 835, the proximal surface of the tissue is punctured at the third fixation point with the delivery needle 20. The delivery needle punctures the proximal surface of the tissue on the proximal side of the defect 4 a second time.

At 840, the distal surface of the tissue is punctured at a fourth fixation point with the delivery needle. The delivery needle 20 punctures the distal surface on the distal side of the defect 4 at the fourth fixation point. A second distance between the second fixation point and the fourth fixation point is between about 5 mm and about 100 mm. The distance between the second fixation point and the fourth fixation point and the edge of the defect is at least 5 mm.

At 845, a second braided anchor segment 9 is extruded from the delivery needle 20. The second braided anchor segment 9 is pushed out of the delivery needle 20 by the second drive tube 70.

At 850, the flexible strand (e.g., suture) 24 is pulled a second time to deform the second braided anchor segment into the second anchor 11. The distal end of the second drive tube 70 is positioned against the proximal end of the second implant 9 to provide resistance against the pulled suture 24.

At 855, the delivery needle is withdrawn from the fourth fixation point and the third fixation point. Following the pulling of the suture 24 and the deforming of the second implant 9, the delivery needle 20 is withdrawn from the third and fourth fixation points, but with the second drive tube 70 still extended. Having the second drive tube 70 extended will protect the suture from the needle edge when pulling the entire construct together.

At 860, the flexible strand is secured with a locking mechanism to keep the tissue edges joined and by fixated by the anchor assembly. After both fixation devices have been placed in the distal side of the defect, the suture outside of the of delivery handle is pulled taut cinches up the mattress stitch that has been placed around the defect. The second drive tube provides resistance to the pulling of the suture 24, and the mattress stitch construct is drawn closed to bring the edges of the defect together in a horizontal seam.

After formation of the horizontal seam, the delivery assembly can be removed from the endoscope, and a locking device (e.g., locking mechanism) such as a cinch or special knot can be pushed down to complete and lock the mattress stitch. Additional locking mechanisms that may be used include a tag, a barb, a cinch, a toggled needle, or distal knot. The remaining suture is cut.

At 865, the flexible strand is cut. The flexible strand is cut a distance away from the fourth fixation point and the locking mechanism. Once the locking mechanism is secure, the suture 24 can be cut to complete the procedure.

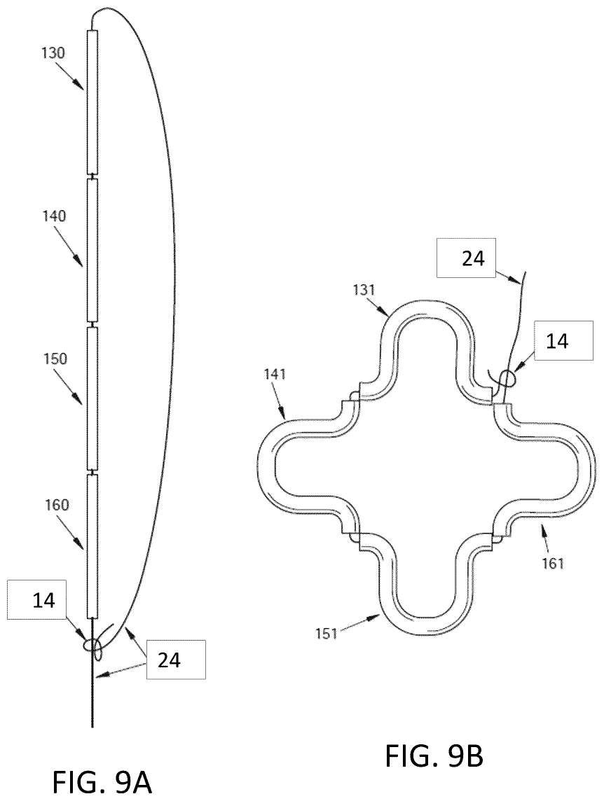

FIG. 9A shows a schematic illustration of an embodiment of braided suture segments on a suture 24. In this embodiment, a series of suture segments are strung on a suture with a slip knot 14 being positioned after the final braided suture segment on the suture 24. FIG. 9A shows a first braided suture segment 130, a second braided suture segment 140, a third braided suture segment 150, and a fourth braided suture segment 160 on a suture 20. In this embodiment, a slip knot 22 is positioned proximal to the fourth braided suture segment 160. That is, in this embodiment, there is no slip knot present at the proximal end of the first braided anchor segment 8, but the slip knot is positioned following the final braided suture segment threaded onto the suture.

In other embodiments, a slip knot or other locking mechanism may be positioned proximally to any segments of an assembly of braided suture segments. That is, a slip knot 22 or other locking mechanism may be positioned proximal to any of suture segments 130, 140, 150, or 160, for example.

FIG. 9B shows a schematic illustration of an assembly of braided anchor segments as they are deployed on the distal side of the defect 4. In this embodiment, the assembly of braided suture segments is only anchored to the tissue at where the slip knot is pulled taut. In this embodiment, the assembly of braided anchor segments has a large surface area that enables a large securing surface with only one hole formed in the tissue. This may be useful in retracting tissue from other tissue and holding it in place while other procedures are being undertaken.

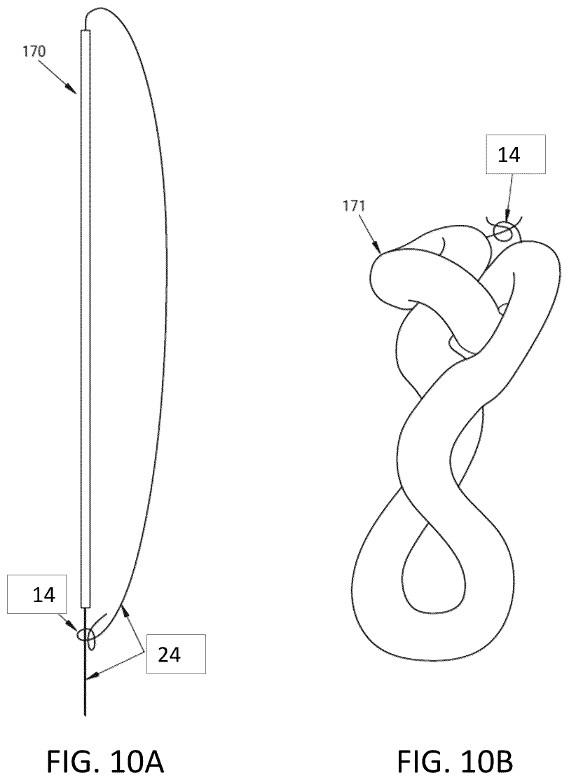

FIG. 10A shows a schematic illustration of an embodiment of a long braided suture segment 170 on a suture 24. In this embodiment the long braided suture segment 170 may be between 40 mm and 70 mm. In this embodiment, a slip knot 22 is positioned proximal to the long braided suture segment 170. In this embodiment, the long braided anchor 170 segment has a large surface area that enables a large securing surface with only one hole formed in the tissue. This may be useful in retracting tissue from other tissue and holding it in place while other procedures are being undertaken.

FIG. 10B shows a schematic illustration of a long braided anchor segment 171 as it is deployed on the distal side of a tissue. In this embodiment, the long braided anchor segment 171 is only anchored to the tissue at where the slip knot 14 is pulled taut. In this embodiment, the long braided anchor segment 171 has a large surface area that enables a large securing surface with only one hole formed in the tissue. This may be useful in retracting tissue from other tissue and holding it in place while other procedures are being undertaken.

In some embodiments, a needle that may be shaped as a straight or curved needle may be used for endoscopic tissue to tissue fixation.

In some embodiments, a method of delivering two implants in order to secure four fixation points includes forming a mattress stitch.

In some embodiments, a method of constructing a mattress stitch includes installing two implants through a tissue tear, creating a mattress stitch on two sides of the tissue tear, cinching the mattress stitch, and locking the construct.

In some embodiments, a sliding knot may be used with a single continuous suture to deform a first implant. A second implant may slidably connect on the suture. The second implant may be used to form a connecting loop whereby a defect can be closed by pulling the suture.

In some embodiments, a sliding knot may be used on a proximal end of a first fixation device to expand and/or deform the device by pulling the suture and utilizing the tail end of the knot to act as a backstop against the delivery tube.

In some embodiments, a tail end of a knot may be used on the proximal end of the fixation device to help prevent the device from pulling through the tissue, and to act as a primary stop.

In some embodiments, a method of using a drive tube to extrude an anchor, and then aid in the deformation of the anchor by pulling against it as a backstop all while using the tube to protect the suture from a needle bevel includes a slidable slip knot positioned between the drive tube and the anchor.

The embodiments of the invention described above are intended to be merely exemplary; numerous variations and modifications will be apparent to those skilled in the art. Such variations and modifications are intended to be within the scope of the present invention as defined by any of the appended claims.

What is claimed is:

1. A method for joining edges of a tissue defect into a seam by implanting an anchor assembly connected via a single flexible strand for fixation of the tissue edges into a seam, the method comprising:
puncturing a proximal surface of a tissue at a first fixation point with a delivery needle, the first fixation point on a proximal side of the tissue defect;
puncturing a distal surface of the tissue at a second fixation point with the delivery needle, the second fixation point on a distal side of the tissue defect;
extruding a first braided anchor segment of an anchor assembly from the delivery needle, the anchor assembly comprising:
the single flexible strand; and
at least two braided anchor segments slidably coupled to the single flexible strand, the single flexible strand passing through a bore hole that traverses from a proximal end to a distal end of the at least two braided anchor segments such that the proximal end of a first braided anchor segment is adjacent to a distal end of a second braided anchor segment;
pulling the flexible strand a first time to deform the first braided anchor segment, the deformed first braided anchor segment configured to prevent the first braided anchor segment from being pulled through the tissue;
withdrawing the delivery needle from the second fixation point and the first fixation point;
approximating the delivery needle at a third fixation point, the third fixation point being a first distance from the first fixation point;
puncturing the proximal surface of the tissue at a third fixation point with the delivery needle; the third fixation point on a proximal side of the tissue defect;
puncturing the distal surface of the tissue at a fourth fixation point with the delivery needle; the fourth fixation point on a distal side of the tissue defect, the fourth fixation point a second distance from the second fixation point;
extruding a second braided anchor segment from the delivery needle;
pulling the flexible strand a second time to deform the second braided anchor segment;
withdrawing the delivery needle from the fourth fixation point and the third fixation point;
pulling the flexible strand a third time to cinch the flexible strand, pulling the flexible strand draws the second fixation point toward the first fixation point and draws the fourth fixation point to the third fixation point to join edges of tissues into a seam;
securing the flexible strand to keep the tissue edges joined and fixated by the anchor assembly; and
cutting the flexible strand, the flexible strand is cut a distance away from the fourth fixation point.

2. The method of claim 1, wherein the extruding the first braided anchor segment is accomplished by advancing a first drive tube to push the first anchor segment out of the delivery needle.

3. The method of claim 1, wherein:
the at least two braided anchor segments comprises a slip knot placed around the flexible strand at the proximal end of the first braided anchor segment; and
the securing the flexible strand comprises locking the flexible strand in place using a locking mechanism comprising at least one of a tag, a barb, a cinch, a toggled needle, or a distel knot.

4. The method of claim 1, wherein the securing the flexible strand comprises a locking device or an overhand knot that can be run down to a fixation point.

5. The method of claim 1, wherein puncturing the distal surface of the tissue at a second fixation point with the delivery needle follows continuously from puncturing the proximal surface of the tissue at the first fixation point with the delivery needle.

6. The method of claim 1, wherein:
the first distance is between about 5 mm and about 20 mm; and
the second distance is between about 10 mm and about 100 mm.

7. The method of claim 1, wherein the first distance is about equal to the second distance.

8. The method of claim 1, wherein a distance between the first fixation point, the second fixation point, the third fixation point, and the edge of the tissue defect is at least 5 mm for each of the fixation points.

9. The method of claim 1, wherein the extruding the second braided anchor segment is performed by pushing the second braided anchor segment with a second drive tube.

10. A surgical device for implanting at least two flexible braided anchor segments via a single flexible strand for fixation of the tissue edges into a seam, the surgical device comprising:
a handle assembly, the handle assembly having a cylindrical bore passing through the handle assembly on a center axis along a length of the handle assembly;
a curved delivery needle positioned in the cylindrical bore of the handle assembly, the curved delivery needle being hollow and having a common center line with the cylindrical bore of the handle assembly, the curved delivery needle comprising an outer tube having a needle tip at a distal end of the curved delivery needle;
at least two braided suture segments slidably coupled to a flexible strand, the flexible strand being translationally threaded through each of the at least two braided suture segments, the at least two braided suture segments positioned in the curved delivery needle;
a first drive tube positioned in the delivery needle and having a common center line with the curved delivery needle, the first drive tube configured to extrude a first braided suture segment of the at least two braided suture segments; and
a second drive tube positioned in the first drive tube and having a common center line with the first drive tube and the curved delivery needle, the second drive tube configured to extrude a second braided suture segment of the at least two braided suture segments.

11. The surgical device of claim 8, wherein the at least two braided suture segments comprises hollow braided suture segments.

12. The surgical device of claim 10, wherein:
the flexible strand comprises a monofilament strand;
the at least two braided anchor segments are slidably coupled to the flexible strand, the flexible strand passing through a bore hole that traverses from a proximal end to a distal end of each of the at least two braided anchor segments such that the proximal end of the first braided anchor segment is adjacent to a distal end of a second braided anchor segment; and
the at least braided suture segments comprises hollow braided suture segments aligned end to end having the monofilament strand threaded through the aligned at least two hollow braided suture segments.

13. The surgical device of claim 12, wherein a column strength of the at least two braided suture segments is increased by application of at least one of heat or a cyanoacrylate coating to each of the segments of the at least two braided suture segments.

14. The surgical device of claim 10, wherein the braided suture segments of the at least two braided suture segments have a length of between about 5 mm and about 50 mm.

15. The surgical device of claim 10, wherein each of the braided suture segments of the at least two braided suture segments has a length of between about 5 mm and about 12 mm.

16. The surgical device of claim 11, wherein a distal end of the flexible strand threaded through the first braided suture segment is looped around the first braided suture segment and knotted around the flexible strand between the first braided suture segment and the first drive tube.

17. The surgical device of claim 10, wherein each of the at least two braided suture segments is treated to increase a rigidity of each of the at least two braided suture segments slidably coupled to the flexible strand.

18. The surgical device of claim 17, wherein the treatment comprises:
heating each of the at least two braided suture segments; or
applying a cyanoacrylate coating to each of the at least two braided suture segments.

19. The surgical device of claim 10, wherein:
the curved delivery needle comprises nitinol, the curved delivery needle being flexible;
the handle assembly further comprises:
an outer sheath, the outer sheath positioned in the cylindrical bore of the handle assembly, wherein the curved delivery needle is slidably positioned in the outer sheath.

20. The surgical device of claim 19, wherein:
when the curved delivery needle is withdrawn into the outer sheath, the curved delivery needle conforms to straight tube; and
when the curved delivery needle is pushed out of the outer sheath, the curved delivery needle returns to its curved shape.
